(19) United States
(12) Patent Application Publication
(10) Pub. No.: US 2011/0160645 A1
(43) Pub. Date: Jun. 30, 2011

(54) Cryo Activated Drug Delivery and Cutting Balloons

(75) Inventors: Derek Sutermeister, Eden Prairie, MN (US); James Anderson, Fridley, MN (US)

(73) Assignee: BOSTON SCIENTIFIC SCIMED, INC., Maple Grove, MN (US)

(21) Appl. No.: 12/939,864

(22) Filed: Nov. 4, 2010

Related U.S. Application Data
(60) Provisional application No. 61/291,616, filed on Dec. 31, 2009.

(51) Int. Cl. A61L 29/16; A61B 18/02; A61M 25/10; A61B 17/32

(52) U.S. Cl. 604/22; 606/21; 604/103.02

(57) ABSTRACT

Physical property changes in materials between body temperature and a cryotreatment temperature are used to benefit auxiliary functional structures on a cryotherapy device. The auxiliary functional structures may be drug delivery coatings, cutting balloon blades, balloon stiffeners or force concentrators.

### CROSS-REFERENCE TO RELATED APPLICATIONS

This application claims the benefit of Application No. 61/291,616, filed Dec. 31, 2009, the entire contents of which is hereby incorporated by reference.

### BACKGROUND OF THE INVENTION

Percutaneous intravascular procedures have been developed for treating atherosclerotic disease in a patient's vasculature. The most successful of these treatments is percutaneous transluminal angioplasty (PTA). PTA employs a catheter having an expansible distal end, usually in the form of an inflatable balloon, to dilate a stenotic region in the vasculature to restore adequate blood flow beyond the stenosis. Other procedures for opening stenotic regions include directional atherectomy, rotational atherectomy, laser angioplasty, stents and the like.

Sometimes following an initially successful angioplasty or other primary treatment restenosis occurs within weeks or months of the primary procedure. Restenosis results at least in part from smooth muscle cell proliferation in response to the injury caused by the primary treatment. This cell proliferation is referred to as “hyperplasia.” Blood vessels in which significant restenosis occurs will typically require further treatment.

A number of strategies have been proposed to treat hyperplasia and reduce restenosis. Previously proposed strategies include prolonged balloon inflation, treatment of the blood vessel with a heated balloon, treatment of the blood vessel with radiation, the administration of anti-thrombotic drugs following the primary treatment, stenting of the region following the primary treatment, the use of drug-eluting stents, use of drug delivery balloons, cutting balloons, cryotherapy systems and the like.

Drug delivery balloons that deliver drug to an internal site upon expansion are known. Some involve perfusion of a drug composition through the balloon wall or from a spongy layer on the balloon wall. Others involve delivery of particulate drug, often carried in a polymer or other excipient to the site.

Delivery of drug from the surface during expansion provides benefits of pushing the drug into the specific tissue to be effected and is especially suited for delivering drugs that prevent restenosis during a dilation of a stenotic lesion. However the delivery technique still suffers from a fundamental conflict between the contradictory needs to deliver an effective dose at the treatment site and to keep the drug adhering to the balloon as it is being manipulated to that site. Techniques to improve drug adhesion, such as formulation with polymers or other excipients or application of protective layers, make it more difficult to effectively deliver an effective dose when the balloon is inflated. Conversely if the drug is applied to the balloon unformulated, or is formulated with a highly soluble excipient, for instance contrast agents such as iopamide, or sugars such as sucrose or mannitol, undesirably high losses and dosage variation can result.

Paclitaxel coated balloons that provide high release rates from the balloon surface have recently been developed. In some cases paclitaxel has been applied directly to the balloon or to a coating placed on the balloon. In other cases paclitaxel has been formulated with an excipient that may be polymer, a contrast agent, a surface active agent, or other small molecules that facilitate adhesion to the balloon and/or release from the balloon upon expansion. The formulations have typically been applied from solution, and may be applied to the entire balloon or to a folded balloon, either by spraying, immersion or by pipette along the fold lines. However the commercial balloons do not yet provide for delivery of predictable amounts of the drug to the tissue at the delivery site nor do they provide for a predictable therapeutic drug tissue level over an extended time period. Nor do they address differences in downstream drug loss due to tracking the device through different anatomies.

Earlier investigations of paclitaxel coated balloons by the applicant have shown that it is desirable to control the morphology of the drug on the balloon, that dihydrate paclitaxel crystalline form facilitates longer tissue residence time, and that the formation of crystalline paclitaxel dihydrate can be controlled by use of vapor annealing of the balloon.

Other devices used to treat stenoses include cutting balloons which provide blades for scoring lesions as they are dilated. Evidence has shown that cutting the stenosis, for example with an angioplasty balloon equipped with a cutting edge, during treatment can reduce incidence of re-stenosis. Additionally, cutting the stenosis may reduce trauma at the treatment site and/or reduce the trauma to adjacent healthy tissue. Cutting blades may also be beneficial additions to angioplasty procedures when the targeted occlusion is hardened or calcified. Thus, angioplasty balloons equipped with cutting edges have been developed in an attempt to enhance angioplasty treatments. These devices have their own difficulties in design because of the added stiffness and the necessary protection for the blades. Balloons with stiffeners or force concentrators that provide for higher pressure inflation or focus the balloon pressure at particular locations also can present problems in delivery because of the added stiffness of the added structures.

Cryotherapy systems which cold-treat a lesion are another well known method of treating stenoses. Cryotherapy methods treat a lesion site in a patient's vasculature or other tissues by cooling the tissues to a temperature in a target temperature range. Cryotherapy treatment prevents or slows reclosure of a lesion following angioplasty and has been implemented in the coronary and/or peripheral vasculature by remodeling the lesion using a combination of dilation and cryogenic cooling. Cryotherapy systems frequently apply cold treatment by inflating a balloon with a cryogen.

There is an ongoing need for improved angioplasty devices and improved methods of treating intravascular stenoses and occlusions.

### SUMMARY OF THE INVENTION

The invention provides novel techniques and structures to solve problems of balloon structures such as drug delivery coatings, cutting balloon blades and balloon stiffeners or force concentrators using physical property changes in the materials between body temperature and a cryotreatment temperature.

In some embodiments the invention pertains to a medical device comprising:

a balloon and

a cryogen introduction system for introducing a cryogen into the balloon,

wherein the balloon comprises an auxiliary functional structure formed of a material composition that is in a soft and flexible state at body temperature and that is in a relatively harder, more frangible and/or less adherent state when cooled at a cooling rate obtainable with said cryogen introduction system to a cryotreatment temperature at which the balloon remains functionally operable.

In some embodiments the material composition of the balloon auxiliary functional structure is characterized by a rubbery state at body temperature a glassy state at the cryotreatment temperature. In some embodiments cryotreatment temperatures are in the range of −30 to about 10° C.

In some embodiments the balloon auxiliary functional structure is a coating that remains adherent and flexible at body temperature, but breaks up and releases at cryotreatment temperature so that the coating can be delivered to a treatment site when the balloon is expanded and cooled.

In some embodiments the auxiliary functional structure comprises a blade or member that stiffens portions of the balloon when the member is in its glassy state. For delivery, the relative softness of the blade or stiffener at body temperature facilitates placement and lesion crossing. When inflated at cryotreatment temperature, the blade, stiffener, or force concentrator becomes sufficiently rigid to operate effectively for its designated function. Upon rewarming to body temperature the auxiliary functional structure again becomes more flexible facilitating removal.

In other aspects the invention can utilize two-way shape memory in which a phase transition occurs at a temperature between body temperature and the cryogen treatment temperature. For instance a shape memory metal wire on a balloon that adopts a coiled configuration at body temperature to compress the balloon and a straight configuration at cryogen temperature to stiffen the balloon or concentrate force. Similarly a cutting blade formed of two-way shape memory can utilize a twisted wrap configuration, optionally with a blade edge laid against the balloon wall, and then adopt a low temperature configuration with the blades in operative position.

The embodiments described herein may be combined with each other and with other features known in the treatment of stenoses.

### BRIEF DESCRIPTION OF THE DRAWINGS

FIG. 1 is a perspective partial cutaway view of a cryogenic balloon catheter system according to the principles of the present invention.

FIG. 2 is a partial cutaway view of a balloon catheter of the system of FIG. 1.

FIG. 3 is a cross-sectional view through the balloon catheter of FIG. 3 taken along lines 3-3.

FIGS. 4a-4c are schematic cross-section depictions of a blood vessel that illustrate a method for treatment using a drug coated cryotherapy balloon.

FIG. 5 shows a perspective view of a cutting balloon in accordance with the invention mounted on a catheter.

FIG. 6 shows a cross sectional view of a cutting balloon with drug coating in accordance with the invention.

FIGS. 7 and 8 depict is a schematically a cross-section of a blood vessel with a cutting balloon in accordance with an alternate embodiment of the invention in body temperature deflated and low temperature inflated configurations, respectively.

### DETAILED DESCRIPTION OF PREFERRED EMBODIMENTS

In some embodiments the inventive device uses a cryotherapy balloon combined with cutting balloon technology and/or with drug delivery balloon technology. This provides a combination of short term and long term treatments that are suited to treat specific stenoses with therapies that reduce restenosis, and at the same time allows for improvement in the delivery of these auxiliary functional technologies.

Non-limiting examples of cryotherapy systems are described in the following patents assigned to CryoVascular Systems, Inc.,
U.S. Pat. No. 7,081,112, titled “Cryogenically enhanced intravascular interventions;”
U.S. Pat. No. 7,060,062, titled “Controllable pressure cryogenic balloon treatment system and method;”
U.S. Pat. No. 6,972,015, titled “Cryosurgical fluid supply;”
U.S. Pat. No. 6,908,462, titled “Apparatus and method for cryogenic inhibition of hyperplasia;”
U.S. Pat. No. 6,811,550, titled “Safety cryotherapy catheter;”
U.S. Pat. No. 6,786,901, titled “Cryosurgical fluid supply;”
U.S. Pat. No. 6,786,900, titled “Cryotherapy methods for treating vessel dissections and side branch occlusion;”
U.S. Pat. No. 6,648,879, titled “Safety cryotherapy catheter;”
U.S. Pat. No. 6,602,246, titled “Cryotherapy method for detecting and treating vulnerable plaque;”
U.S. Pat. No. 6,514,245, titled “Safety cryotherapy catheter;”
U.S. Pat. No. 6,468,297, titled “Cryogenically enhanced intravascular interventions;”
U.S. Pat. No. 6,432,102, titled “Cryosurgical fluid supply;”
U.S. Pat. No. 6,428,534, titled “Cryogenic angioplasty catheter;”
U.S. Pat. No. 6,355,029, titled “Apparatus and method for cryogenic inhibition of hyperplasia;”
   and additionally in the following documents,
U.S. Pat. No. 7,604,631, Reynolds (Boston Scientific Scimed, Inc.), titled “Efficient controlled cryogenic fluid delivery into a balloon catheter and other treatment devices;”
US 20090299356, Watson (Boston Scientific Scimed, Inc.), titled “Regulating internal pressure of a cryotherapy balloon catheter;”
US 20090299355, Bencini et al (Boston Scientific Scimed, Inc.), titled “Electrical mapping and cryo ablating with a balloon catheter;”
US2009/0088735, Abboud, et al, (Cryocath Technologies, Inc), titled “Method and Apparatus for Inflating and Deflating Balloon Catheters;”
US 20080208182, Lafontaine, et al, (Boston Scientific Scimed, Inc.), titled “Method for Tissue Cryotherapy.”
   All of these documents are also incorporated herein by reference in their entirety.

A number of different stiffening, cutting, and scoring configurations have been proposed in the art. These include:

U.S. Pat. No. 4,796,629;

U.S. Pat. No. 5,320,634;

U.S. Pat. No. 5,616,149;

U.S. Pat. No. 5,102,402;

U.S. Pat. No. 6,730,105

U.S. Pat. No. 6,942,680

U.S. Pat. No. 7,070,576;

U.S. Pat. No. 7,303,572;

U.S. Pat. No. 7,604,631;

U.S. Pat. No. 7,632,288

US 2002/0010489;

US 2003/0153870;

US 2004/143287;

US 2005/288629;

US 2006/0129093;

US 2009/0105687; and

US 2009/0192537.

All of these documents are incorporated herein by reference in their entirety.

Drug delivery balloon systems are described in the following documents:
U.S. Pat. No. 5,102,402, Dror et al (Medtronic, Inc.);
U.S. Pat. No. 5,370,614, Amundson et al, (Medtronic, Inc.);
U.S. Pat. No. 5,954,706, Sahatjian (Boston Scientific Corp);
WO 00/32267, SciMed Life Systems; St Elizabeth's Medical Center (Palasis et al);
WO 00/45744, SciMed Life Systems (Yang et al);
R. Charles, et al, “Ceramide-Coated Balloon Catheters Limit Neointimal Hyperplasia After Stretch Injury in Cartoid Arteries,” Circ. Res. 2000; 87; 282-288;
U.S. Pat. No. 6,306,166, Barry et al, (SciMed Life Systems, Inc.);
US 2004/0073284, Bates et al (Cook, Inc; MED Inst, Inc.);
US 2006/0020243, Speck;
WO 2008/003298 Hemoteq AG, (Hoffman et al);
WO 2008/086794 Hemoteq AG, (Hoffman et al);
US 2008/0118544, Wang;
US 20080255509, Wang (Lutonix); and
US 20080255510, Wang (Lutonix),
   and in the following US provisional applications:
61/172,629, filed Apr. 24, 2009, entitled “Use of Drug Polymorphs to Achieve Controlled Drug Delivery From a Coated Medical Device;”
61/224,723, filed Jul. 10, 2009, entitled “Use of Nanocrystals for a Drug Delivery Balloon; and
61/271,167, filed Jul. 17, 2009, entitled “Nucleation of Drug Delivery Balloons to Provide Improved Crystal Size and Density.”
   All of these documents are also incorporated herein by reference in their entirety.

Cryotherapy systems to which the invention pertains include a cryogenically cooled balloon, control systems for inflating and cryogenically cooling tissue at a treatment site, e.g. at a vascular stenosis. Balloon designs, control systems, and techniques are described in detail in documents listed above. Cryotherapy systems allow a wide variety of temperature and/or pressure treatment profiles and include techniques to inflate balloons at least in part without therapeutic cooling. The use of cooling before and/or during dilation of a lesion may allow the use of dilation balloon inflation pressures which are lower than those typically applied for uncooled balloon angioplasty.

In some embodiments, inflating cryotherapy balloon at a pressure, for instance of about 8 atm or more, and cooling the engaged vessel wall tissues to a temperature between about 0° C. to about −20° C., for instance in the range of −2° C. to −12° C., can open a stenotic lesion while inhibiting recoil and/or restenosis. Cryoablation systems that treat at temperatures considerably lower, for instance −60° C. to −90° C. are also known.

The invention builds on these known cryotreatment systems by taking advantage of the temperature change at the treatment site to change the property of an auxiliary functional structure on the balloon. In some embodiments the auxiliary functional structure is formed of a composition that changes its physical properties from a relatively softer or rubbery material to a substantially harder, more frangible and/or less adherent form.

In some embodiments the auxiliary functional structure on the balloon is a drug coating, in others it is a cutting blade, a balloon stiffener or a force concentrator.

In at least some embodiments the auxiliary functional structure can be more effectively operated to perform its designated function at the cryotherapy temperature than at body temperature. In some embodiments the auxiliary functional structure will not be operable to perform its designated function at body temperature but is more easily or effectively delivered to the treatment site at body temperature and is functionally operable when cooled to the cryotherapy temperature.

In the case of a drug delivery coating, the reduced temperature provides the coating with lower adhesion to substrate. In some embodiments the coating composition is specifically formulated to become frangible at the cryotreatment temperature so that upon balloon expansion it fractures and loosens allowing the drug coating to be delivered at the site more reliably. In other embodiments a drug layer, for instance a drug particle layer is protected by a polymer overlayer that fragments at the cryo treatment temperature to release the drug particles. According to the invention many options in formulation of drug coatings or protective coating layers are made available because the objectives of providing coating integrity during manipulation to the site and rapid release at the site are not intrinsically contradictory when release at the site is facilitated by a substantial reduction in temperature.

In some embodiments the drug is delivered in a formulation that provides for extended release into adjacent tissue. While this possibility has been recognized previously for drug delivery balloons, the problems of the inefficiency of delivery have significantly limited the design options for extended release formulations on drug delivery balloons.

The invention also provides substantial improvements for cutting balloons and balloons utilizing stiffening structures. For instance, if a cutting blade is rubbery at body temperature, the device has an improved margin of safety and ease of delivery. In some cases a different fold profile can be implemented or refold profile becomes less critical because the blade is rubbery during delivery and recovery. However when inflated and cooled at the treatment site the blade becomes sufficiently rigid to score a lesion, for instance a calcified lesion.

Materials change their physical properties with temperature at different rates. Polymer materials in particular often stiffen substantially at low temperatures. In many of these cases there may be a detectable glass transition that occurs at some point in which a material changes from a relatively rubbery condition to a more rigid glass-like material. At least some embodiments of the invention exploit such a glass transition. In particular the material used to form the auxiliary functional balloon structure is one that undergoes a glass transition in the range between body temperature and the cryotreatment temperature. In specific embodiments the treatment temperature is in the range of from about −30° C. to about 10° C. and the material of the auxiliary functional structure on the balloon undergoes a glass transition between body temperature and the treatment temperature.

In this context it should be recognized that glassy behavior of a material can be influenced by the rate of cooling or heating. Generally the faster a material is cooled the more rigid it appears at a given temperature and the glass transition will be apparent at a higher temperature. Glass transition for purposes of the invention should be taken at a cooling rate obtainable with the cryogen introduction system and preferably at a range of cooling rates reflective of those specifically contemplated for implementation as treatment protocols for the device. Of course the glass transition of the auxiliary functional structure on the balloon should occur at a temperature at which the balloon remains functionally operable. In specific embodiments the auxiliary functional structure on the balloon is formed of a material that undergoes glass transition above that contemplated for implementation in the treatment protocol. In some embodiments glass transition is measured by differential scanning calorimetry.

It should be noted that in other embodiments, a sufficient difference in physical properties may be provided by cooling a auxiliary functional structure formed of a material that undergoes no detectable glass transition in the temperature range from body temperature to the cryotreatment temperature. For instance, in the case of a polymeric balloon stiffener or blade, even if the material selected for such auxiliary functional structure is already below its glass transition temperature at body temperature, there may still be enough of an increase in stiffness at the cryotreatment temperature to allow for thinner stiffeners to be utilized. This will make the balloon more flexible at body temperature, and hence more easily delivered to the treatment site.

In some embodiments the material is formulated to undergo an increase in flexural modulus of about 15% or more, about 20% or more, about 30% or more, or more or about 50% or more, for instance 20%-60% or 30%-50%, between body temperature and the cryotreatment temperature. In some embodiments a substantial change in flexural modulus of about 30% or more is preferred.

If a particular polymer does not have sufficient flexibility at body temperature it may be blended with a second polymer or with a plasticizer to provide the desired properties. Conversely if a polymer has a glass transition that is too low it may be blended with a second with a higher Tg polymer, or it may be crosslinked, or in some cases mixed with a filler.

In specific embodiments the auxiliary functional structure is formed of a material composition that comprises a polymer. The polymer material may be thermoplastic or crosslinked, may be branched or linear, and may contain additives such as plasticizers or fillers, all of which can affect the glass transition behavior of the material.

In embodiments of the invention in which the auxiliary functional structure comprises a drug coating or protective coating, the coating changes its physical properties because of a cold treatment at the site which is provided by the cryotherapy system.

In some embodiments the drug containing layer is applied over an underlayer of material that has a high solubility in bodily fluids to undercut the drug and facilitate breakup of the drug-containing layer upon balloon expansion. An example of a suitable underlayer material is pectin.

For a drug coating the drug in some embodiments will be formulated with a polymeric carrier. The drug may be dissolved or dispersed in the polymeric carrier. Other additives such as plasticizers, fillers or surfactants may also be included in the coating material. The drug coating or protective coating changes its physical properties by reason of a cold treatment at the site which is provided by the cryotherapy system if the drug composition and/or a protective layer over the drug, are formulated to have a glass transition in the range between body temp and the cryotreatment temperature.

For purposes of the invention the term drug includes both therapeutic agents and diagnostic agents. Non-limiting examples of other drugs that may be employed include anti-restenosis agents, antiproliferative agents, antibiotic agents, antimitotic agents, antiplatelet agents, alkylating agents, platinum coordination complexes, hormones, anticoagulants, fibrinolytic agents, antimigratory agents, antisecretory agents, anti-inflammatory agents, indole acetic acids, indene acetic acids, immunosuppressive agents, angiogenic agents, angiotensen receptor blockers, nitric oxide donors, anti-sense oligonucleotides, cell cycle inhibitors, mTOR inhibitors, growth factor receptor signal inhibitors, transduction kinase inhibitors, retenoids, cyclin/CDK inhibitors, HMG co-enzyme reductase inhibitors, protease inhibitors, viral gene vectors, macrophages, monoclonal antibodies, x-ray contrast agents, MRI contrast agents, ultrasound contrast agents, chromogenic dyes, fluorescent dyes, and luminescent dyes. In some embodiments the drug is a lipophilic substantially water insoluble drug, such as paclitaxel, rapamycin (also known as sirolimus), everolimus, zotarolimus, biolimus A9, dexamethasone, tranilast or another drug that inhibits restenosis. Other drugs that may be suitable are described in the documents incorporated elsewhere herein. Mixtures of drugs, for instance two or more of paclitaxel, rapamycin, everolimus, zotarolimus, biolimus A9, dexamethasone and/or tranilast may be employed.

The drug may be one that has polymorph forms, i.e. at least two characterizable morphologies that have different solubilities, or crystal forms. In some embodiments the different morphological forms have characteristics that affect tissue uptake of the drug at the delivery site. Drugs such as paclitaxel have more than one such morphological form. These have different solubilities and dissolution rates in body fluids, including blood. For some embodiments the drug is provided in a specific polymorph form(s) or distribution of such forms to facilitate a particular theraupetic objective. In some cases the drug also is provided in a particulate size profile that facilitates uptake by the adjacent tissue rather than dissolving into the blood stream and some fraction taken up by the vessel (the therapeutic dose). Very small particles, <1 μm, can be taken up directly into the arterial tissue. Some of the drug that diffuses into the vessel wall binds to and stabilizes the cell microtubules, thereby affecting the restenotic cascade after injury of the artery.

In exemplary embodiments a drug coating on a balloon comprises dose density of between 0.25/mm² and 5 μg/mm² of a drug, for instance paclitaxel, rapamycin, everolimus, zotarolimus, biolimus A9, dexamethasone and/or tranilast.

In some embodiments of a paclitaxel containing drug coating, the fraction of the paclitaxel in the coating that is amorphous is from 0-25%, for instance about 1% to about 5%, based on total paclitaxel weight. In some embodiments the fraction of the paclitaxel in the coating that is anhydrous from 0% to about 99%, for instance 5-95%, about 10%, about 15%, about 20%, about 25%, about 30%, about 40%, about 50%, about 60%, about 70%, about 70%, or about 80%, based on total paclitaxel weight. In some embodiments the fraction the paclitaxel in the coating that is dihydrate crystalline is from 1% to 100%, for instance 1-99%, 5-95%, about 10%, about 15%, about 20%, about 25%, about 30%, about 35%, about 40%, about 45%, about 50%, about 55%, about 60%, about 65%, about 70%, about 75%, about 80%, about 85%, about 88%, about 89%, about 90%, about 91%, about 92%, about 93%, about 94%, about 95%, about 96%, about 97%, about 98%, or about 99%, based on total paclitaxel weight.

In some embodiments the drug in a drug coating is in a particulate form that has a particle size in the range of 0.01-20.0 μm (10-20000 nm). Multi-modal ranges, prepared, e.g. by mixing two or more sets of different narrow size range may be used in some cases to provide a desired bioavailability profile over time. For example 50% of the crystals can be of 1000 nm mean size and the other 50% could be 300 nm mean size. These embodiments enable a tailoring of the drug persistence in the vessel wall. The smaller crystals will more readily dissolve and enter the tissue for immediate effect and larger crystals will dissolve at a much slower rate enabling longer drug persistence. In some embodiments the drug particles may take the form of microcapsules (i.e. the drug particle does not include an encapsulant enclosing the drug), which are in turn mixed with a polymeric carrier to form a drug coating. Paclitaxel crystalline dihydrate is exemplary of a suitable sized particulate drug.

Particular embodiments of the invention use one or more biodegradable polymers in a coating composition. Biodegradable polymers include polyesters, poly(amino acids), copoly(ether-esters), polyalkylenes oxalates, polyamides, poly(iminocarbonates), polyorthoesters, polyoxaesters, polyamidoesters, polyoxaesters containing amido groups, poly(anhydrides), polyphosphazenes, poly-α-hydroxy acids, trimethylene carbonate, poly-β-hydroxy acids, polyorganophosphazines, polyesteramides, polyethylene oxide, polyester-ethers, polyphosphoester, polyphosphoester urethane, cyanoacrylates, poly(trimethylene carbonate), poly(iminocarbonate), polyalkylene oxalates, polyvinylpyrolidone, polyvinyl alcohol, poly-N-(2-hydroxypropyl)-methacrylamide, polyglycols, aliphatic polyesters, poly(orthoesters), poly(ester-amides), polyanhydrides, polysaccharides, and proteins. Specific examples include polyhydroxyalkanoates (PHA), polyhydroxybutyrate compounds, and co-polymers and mixtures thereof, poly(glycerol-sebacate), polypeptides, poly-α-hydroxy acid, such as polylactic acid (PLA). PLA can be a mixture of enantiomers typically referred to as poly-D,L-lactic acid. Alternatively, the biodegradable material is poly-L(+)-lactic acid (PLLA) or poly-D(−)-lactic acid (PDLA), which differ from each other in their rate of biodegradation. PLLA is semicrystalline. In contrast, PDLA is amorphous, which can promote the homogeneous dispersion of an active species. Other examples include polyglycolide (PGA), copolymers of lactide and glycolide (PLGA), polydioxanone, polygluconate, polylactic acid-polyethylene oxide copolymers.

In the case of drug coatings it should be noted that the drug will likely affect the glass transition properties of the polymer carrier, either acting as a plasticizer or a filler and should be accounted for accordingly.

In drug coating embodiments if a plasticizer is used it suitably is a biodegradable plasticizer. Examples of suitable biodegradable plasticizers that may be employed include citrate esters, for instance tributyl citrate, triethyl citrate, acetyltributyl citrate, and acetyltriethyl citrate; polyols, such as glycerin, polyglycerin, sorbitol, polyethylene glycol and polypropylene glycol; starches; vegetable oils; glucose or sucrose ethers and esters; polyethylene glycol ethers and esters; low toxicity phthalates; alkyl phosphate esters; dialkylether diesters; tricarboxylic esters; epoxidized oils; epoxidized esters; polyesters; polyglycol diesters; aliphatic diesters, for instance dibutyl sebacate; alkylether monoesters; dicarboxylic esters; lecithin; and/or combinations thereof. If the composition is hydrophilic, the possible plasticizing effects of exposure to body fluids during delivery should also be taken into account.

Numerous other excipients and additive compounds, protective polymer layers, underlayer materials and drugs are described in one or more of the documents incorporated herein by reference.

In some embodiments a drug may be coated with a protective polymeric layer that functions to reduce loss during deployment of the device to the site of administration, but that substantially disintegrates in the course of the deployment or during transfer of the drug from the device at the site of administration. Suitably such protective layer has a thickness of 1 μm or less, 0.5 μm or less, or 0.1 μm or less. Polymers or copolymers that have a good solubility in water or blood and a molecular weight sufficient to slow dissolution of the coating enough to provide practical protection may be used. Protective layers will suitably be effective if they break up into fine particles during drug delivery, for instance upon balloon expansion. The invention facilitates such breakup. Protective coating thickness may be adjusted to give an acceptable dissolution and/or degradation profile.

In some embodiments the drug is formulated with an excipient. An excipient is an additive to a drug-containing layer that facilitates adhesion to the balloon and/or release from the balloon upon expansion. The excipient may be polymer, a contrast agent, a surface active agent, or other small molecule. In at least some embodiments the drug is substantially insoluble in the excipient.

In some embodiments the excipient substantially degrades or dissolves in the course of the deployment or during transfer of the drug from the device at the site of administration such that little or none of the excipient is detectable on the tissue after a short interval, for instance an interval of 2 days, 1 day, 12 hours, 4 hours, 1 hour, 30 minutes, or 10 minutes.

According to the invention, formulation with excipients or carriers or protective coatings is made much easier because the physical properties of adhesion during manipulation to the site and rapid release at the site are much more easily provided when two different temperatures are used for delivery of the device to the treatment site and for release of the coating composition from the device.

Non-limiting examples of the invention are illustrated in the Figures.

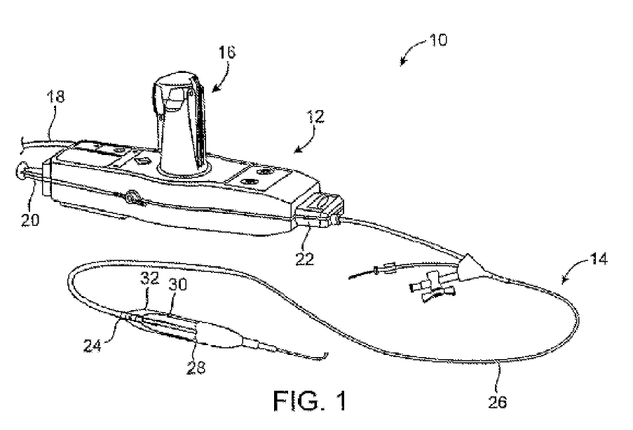

Referring now to FIG. 1, a catheter system 10 generally includes a controller/supply unit 12 and a catheter 14. Unit 12 includes a cooling fluid supply 16 along with cooling fluid control system components such as valves, pressure transducers, electronic controller hardware and/or software, and the like. Unit 12 may optionally incorporate user interface capabilities including switches, input keys, a display, and the like. Alternative embodiments may make use of external user interface or data processing structures, and the components of unit 12 may be separated into different housing structures. The exemplary supply/control unit 12 also includes a cable 18 for supplying electrical power from a battery, wall outlet, or other convenient power source, which may alternatively be provided from an internal power source.

A vacuum source 20 is integrated into unit 12, here in the form of a positive displacement pump such as a syringe. A housing of unit 12 has a size, shape, and weight suitable for holding in a single hand during a procedure. Unit 12 is coupled to catheter 14 by interfacing hubs or connectors 22 on the unit and catheter.

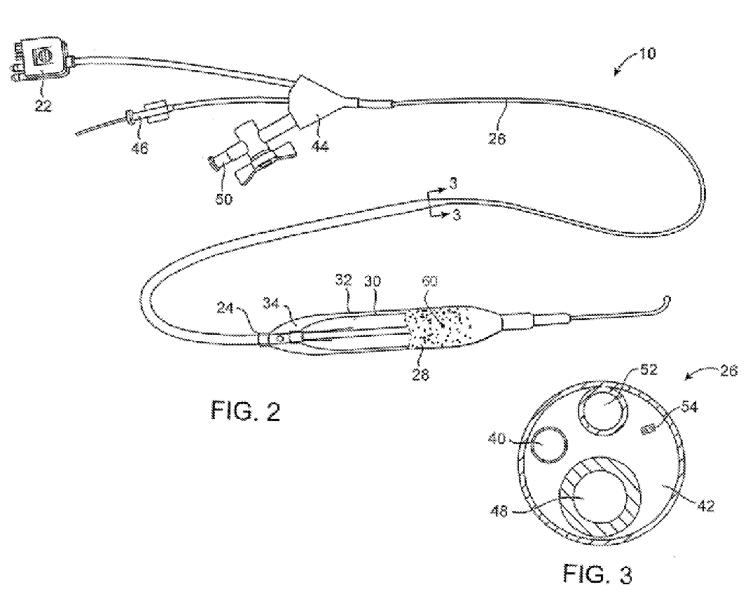

Catheter 14 generally has a proximal end adjacent connector 22, a distal end 24, and an elongate catheter body 26 extending therebetween. A balloon 28 is disposed adjacent to the distal end 24 of catheter body 26. In the exemplary embodiment, balloon 28 comprises an inner balloon 30 and an outer balloon 32 with a vacuum space (see FIG. 2). By monitoring a vacuum applied between the first and second balloons, and by shutting off the cooling fluid flow if the vacuum deteriorates, containment of both the first and second balloons can be effectively monitored and release of cooling liquid or gas within the vasculature can be inhibited.

For cryogenic inflation of a balloon, the inflation fluid, for instance nitrous oxide, may be maintained in a canister within unit 12 at a high pressure.

A variety of control methodologies may be employed to control the balloon inflation rate, including any of those more fully described in documents incorporated herein.

Unit 12 may be selectively coupled to any of a plurality of selectable balloon catheters, which will often have catheter bodies, balloons, and/or other components with significantly differing characteristics. More specifically, an exemplary set of alternatively selectable catheters may include differing combinations of catheter body lengths, flow characteristics, balloon diameters, and/or orifice lengths. Suitably, a control methodology providing a controlled inflation rate for any of the selected balloon catheters when coupled to unit 12, is utilized. In some embodiments the system may be provided with a system for recirculation of coolant, also as known in the art.

Referring now to FIGS. 2 and 3, catheter body 26 includes a cooling fluid supply lumen 40 and an exhaust lumen 42 extending the proximal and distal ends of the catheter body. The balloon 28 is comprised of first and second balloon members 30, 32 may be integral extensions of the catheter body, or may be separately formed and attached thereto. The balloon members 30, 32 may be formed from the same or different material as the catheter body and may be attached to the catheter body by adhesives, heat welding, or the like. Catheter body 26 may comprise a variety of polymer materials, including polyethylenes, polyimides, nylons, polyesters, and/or copolymers and derivatives thereof.

The balloon members 30, 32, respectively of balloon 28, may be elastic and/or inelastic balloons, and may be formed of material such as nylon, polyethylene terephathalate (PET), urethane, latex, silicone, polyethylene, high strength polymers such as Pebax®, and/or the like. Balloon members 30, 32 may be formed from different materials, for example, the first balloon comprising a high-strength material such as PET, while the second balloon comprising a highly durable material such as polyethylene. polyethylene terephthalate (PET), polyetherimide (PEI), polyethylene (PE), etc. Some other examples of suitable polymers, may include polytetrafluoroethylene (PTFE), ethylene tetrafluoroethylene (ETFE), fluorinated ethylene propylene (FEP), polyoxymethylene (POM), polybutylene terephthalate (PBT), polyether block ester, polyurethane, polypropylene (PP), polyvinylchloride (PVC), polyether-ester (for example, a polyether-ester elastomer such as ARNITEL® available from DSM Engineering Plastics), polyester (for example, a polyester elastomer such as HYTREL® available from DuPont), polyamide (for example, DURETHAN® available from Bayer or CRISTAMID® available from Elf Atochem), elastomeric polyamides, block polyamide/ethers, polyether block amide (PEBA, for example, available under the trade name PEBAX®), silicones, Marlex high-density polyethylene, Marlex low-density polyethylene, linear low density polyethylene (for example, REXELL®), polyetheretherketone (PEEK), polyimide (PI), polyphenylene sulfide (PPS), polyphenylene oxide (PPO), poly(ethylene naphthalenedicarboxylate) (PEN), polysulfone, nylon, perfluoro(propyl vinyl ether) (PFA), other suitable materials, or mixtures, combinations, copolymers thereof, polymer/metal composites, and the like.

In some embodiments both balloon members 30, 32 are formed from Pebax® polymers, suitably Pebax® 6333, Pebax® 7033, Pebax® 7233 or a mixture thereof. PEBA polymers such as the Pebax® polymers, which are ester-linked polyamide-block-ethers have a operable range well below −30° C. so they have particular utility as a balloon material in the implementation of the invention.

Balloon 28 will typically have a length of at least 1 cm, preferably being in a range from about 1.5 cm to 20 cm, and may have diameters in a range from 1.5 mm to about 40 mm.

A thermal barrier may be disposed within vacuum space 34, the thermal barrier comprising or maintaining a gap between the balloons. Suitable thermal barriers may comprise woven, braided, helically wound, or knotted fibers such as polyester material. A radiopaque marker may also be disposed on the polyester layer, or otherwise between the first and second balloons so as to facilitate imaging.

Still referring to FIGS. 2 and 3, a hub 44 along catheter body 26 may couple a guidewire port 46 to a guidewire lumen 48 of the catheter body. A balloon deflation port 50 is coupled to exhaust lumen 42 so as to facilitate deflation of the balloon after completion of a procedure. At least one rupture disk may be disposed between the inner surface of the inner balloon and the vacuum space so as to shut down the system prior to a balloon burst. Vacuum space 34 may be coupled to hub 22 by vacuum lumen 52, while wire 54 couple sensors of the balloon to unit 12.

The balloon 28 has a drug coating 60 thereon in accordance with the invention.

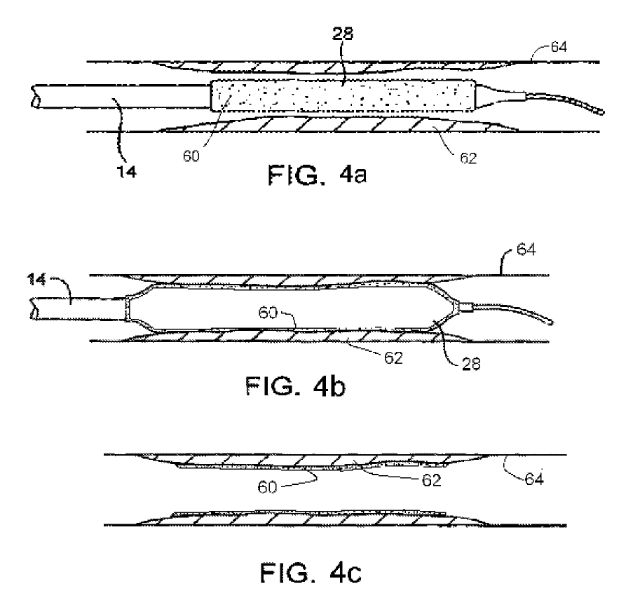

Referring now to FIGS. 4a to 4c, a method for treating a target lesion 62 of a blood vessel 64 can be understood. In FIG. 4a catheter 14 been introduced at body temperature over a guidewire so that balloon 28 is positioned within the blood vessel adjacent the target lesion 64. The coating 60 is formulated to be adherent at body temperature so that the delivery to the lesion 64 does not substantially degrade the coating.

In FIG. 4b the balloon has been expanded with cryogenic cooling bringing the balloon coating 60 into contact with the target lesion 64 at the same time it becomes glassy and non-adherent.

In FIG. 4c, after the balloon has been removed the drug coating 60 is left behind in contact with the lesion.

Cryogenic cooling may be pulsed or continuous and the length of pulses and pulse intervals may very in accordance with known cryotreatment methods. In some embodiments cryotreatment may not be the major objective, in which case the cooling conditions may be fitted to optimize delivery of the coating 60.

In accordance with some embodiments of the invention the auxiliary balloon structure is one or more cutting blades made of a polymer material that is soft at body temperature and will not score a calcified lesion. However when the balloon is inflated with coolings the material stiffens sufficiently to function effectively. Upon return to body temperature the blade material substantially softens again allowing for safer and easier removal. In some embodiments the blade material is formulated to undergo an increase in flexural modulus of about 15% or more, about 20% or more, about 30% or more, or more or about 50% or more, for instance 20%-60% or 30%-50%, between body temperature and the cryotreatment temperature. Upon returning to body temperature the material will decrease in modulus, preferably to approximately its starting body temperature modulus.

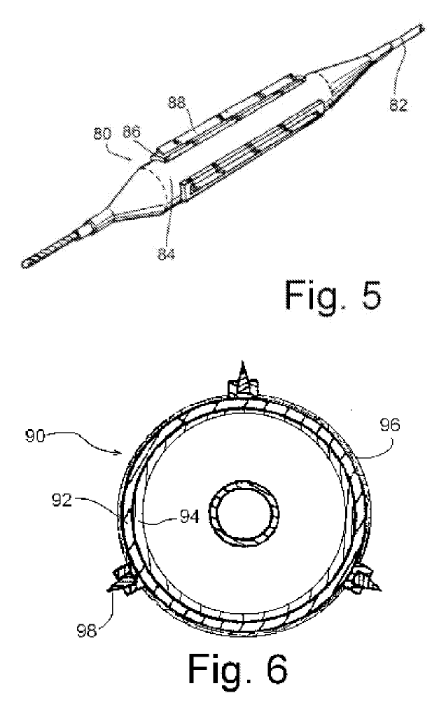

Referring to FIG. 5 there is depicted a cutting balloon designated generally at 80 mounted on a catheter 82. The catheter is equipped with cryogen supply to inflate the balloon 80. Balloon 80 includes a body portion 84, cutting blade mounts 86 and cutting blades 88. The cutting blade 88 and the cutting blade mounts 86 are formed of polymeric materials. When at body temperature, at least the cutting blade 88, and optionally the mounts 86, are above the glass transition of their respective polymeric materials. When cooled to a cryotreatment temperature, the material of the blade is below its glass transition temperature and sufficiently rigid to score calcified lesions. In some embodiments a balloon 80 may include a drug coating on the body portions 84, the mounts 86 or the cutting blades 88 that breaks up for delivery at cryotreatment temperature.

Referring to FIG. 6 there is shown a cross sectional view of a cutting balloon 90 inflated at a cryotreatment temperature. The balloon 90 is comprised of first and second balloon members 92 and 94, a coating 96 on the body portion between cutting blades 98. The inflation of the balloon at cryotreatment temperature has caused the coating 96 to fracture and loosen from the balloon.

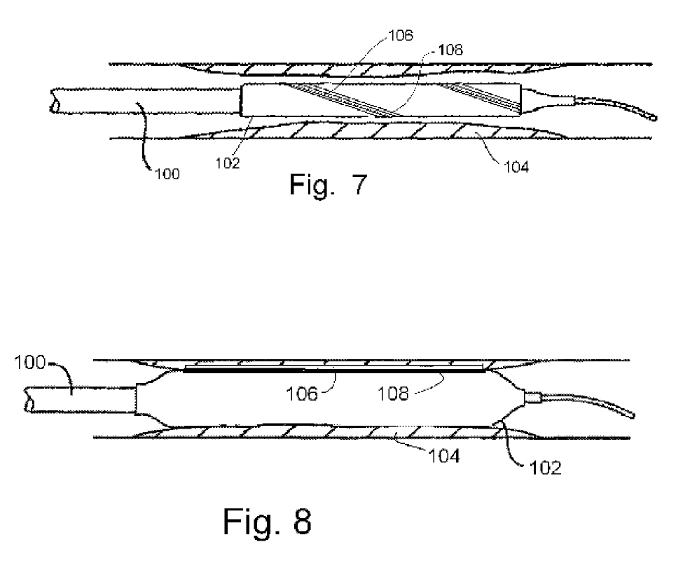

In other aspects the invention can utilize two-way shape memory in which a phase transition occurs at a temperature between body temperature and the cryogen treatment temperature. For instance, a blade stiffener or force concentrator may be mounted on a balloon to adopt a coiled configuration at body temperature to compress the balloon and a straight configuration at cryogen temperature to stiffen the balloon or provide a cutting blade. FIGS. 7 and 8 illustrate this aspect of the invention.

FIG. 7 schematically depicts a catheter 100 with a wrapped balloon 102 at a site 104 of a lesion. Balloon 102 contains a cutting blade 106 held in place by mounts 108. The blade is in a body temperature configuration with the blade 106 laid over sideways. Cryoinflation of the balloon causes the blade to adopt a low temperature memory configuration in which the blade 102 adopts a conventional straight orientation and presses radially into the lesion 104, as shown in FIG. 8. Evacuating the balloon retracts it from the lesion and warming back to body temperature restores a helically wrapped configuration similar to FIG. 7.

In various embodiments one or more cutting members or blades may be coupled to the balloon. The balloon may include one or more discrete points or areas of flexibility to enhance flexibility of the cutting balloon catheter. A break in the one or more cutting members may be aligned with the one or more discrete points of flexibility in the balloon.

In still other embodiments the auxiliary functional structure is a stiffener or force concentrator that is similarly rubbery at body temperature but functionally operative at the cryotreatment temperature.

In the case of a polymeric cutting blade, balloon stiffener, or force concentrator, the polymer used need not be biodegradable and may be any one that can be safely inserted into the body for the requisite period of treatment. The same is true for any plasticizers, fillers and other additives used.

In still another embodiment the auxiliary functional structure may be an adhesive layer between the balloon and a stent. The adhesive is functional at body temperature, retaining the stent on the balloon as it tracked to the delivery site, but becomes non-adherent or frangible at the cryotherapy temperature so that upon cooling and balloon expansion, the adhesive fails, adhesively and/or cohesively, releasing the stent from the balloon.

The devices of the present invention, may be deployed in vascular passageways, including veins and arteries, for instance coronary arteries, renal arteries, peripheral arteries including illiac arteries, arteries of the neck and cerebral arteries, and may also be advantageously employed in other body structures, including but not limited to arteries, veins, biliary ducts, urethras, fallopian tubes, bronchial tubes, the trachea, the esophagus and the prostate.

All published documents, including all US patent documents, mentioned anywhere in this application are hereby expressly incorporated herein by reference in their entirety. Any copending patent applications, mentioned anywhere in this application are also hereby expressly incorporated herein by reference in their entirety.

The above examples and disclosure are intended to be illustrative and not exhaustive. These examples and description will suggest many variations and alternatives to one of ordinary skill in this art. All these alternatives and variations are intended to be included within the scope of the claims, where the term “comprising” means “including, but not limited to”. Those familiar with the art may recognize other equivalents to the specific embodiments described herein which equivalents are also intended to be encompassed by the claims. Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all claims which possess all antecedents referenced in such dependent claim if such multiple dependent format is an accepted format within the jurisdiction. In jurisdictions where multiple dependent claim formats are restricted, the following dependent claims should each be also taken as alternatively written in each singly dependent claim format which creates a dependency from an antecedent-possessing claim other than the specific claim listed in such dependent claim.

1. A medical device comprising
a balloon and
a cryogen introduction system for introducing a cryogen into the balloon,
wherein the balloon comprises an auxiliary functional structure formed of a material composition that is in a soft and flexible state at body temperature and that is in a relatively harder, more frangible, and/or less adherent state when cooled at a cooling rate obtainable with said cryogen introduction system to a cryotreatment temperature at which the balloon remains functionally operable.

2. A medical device as in claim 1 wherein said cryotreatment temperature is in the range of from about −30° C. to about 10° C.

3. A medical device as in claim 1 wherein
the balloon is expandable at a site within the body of a subject to be treated,
said auxiliary functional structure comprises a coating on an outer surface thereof comprising a drug, and
the coating is adapted to be adherent to the balloon surface at body temperature but to at least partially release therefrom when the balloon is cooled and expanded.

4. A medical device as in claim 3 wherein said drug comprises at least one member from the group consisting of anti-restenosis agents, antiproliferative agents, antibiotic agents, antimitotic agents, antiplatelet agents, alkylating agents, platinum coordination complexes, hormones, anticoagulants, fibrinolytic agents, antimigratory agents, antisecretory agents, anti-inflammatory agents, indole acetic acids, indene acetic acids, immunosuppressive agents, angiogenic agents, angiotensen receptor blockers, nitric oxide donors, anti-sense oligonucleotides, cell cycle inhibitors, mTOR inhibitors, growth factor receptor signal inhibitors, transduction kinase inhibitors, retenoids, cyclin/CDK inhibitors, HMG co-enzyme reductase inhibitors, protease inhibitors, viral gene vectors, macrophages, monoclonal antibodies, x-ray contrast agents, MRI contrast agents, ultrasound contrast agents, chromogenic dyes, fluorescent dyes, and luminescent dyes.

5. A medical device as in claim 3 wherein said drug comprises at least one member of the group consisting of paclitaxel, rapamycin, everolimus, zotarolimus, biolimus A9, dexamethasone and tranilast.

6. A medical device as in claim 5 wherein said drug comprises paclitaxel dihydrate in particulate form.

7. A medical device as in claim 6 wherein at least a portion of the paclitaxel dihydrate in particulate form has a particle size of less than 1 μm.

8. A medical device as in claim 3 where said drug coating further comprises a polymer.

9. A medical device as in claim 8 wherein said polymer is biodegradable.

10. A medical device as in claim 8 wherein said polymer is a member of the group consisting of polyesters, poly(amino acids), copoly(ether-esters), polyalkylenes oxalates, polyamides, poly(iminocarbonates), polyorthoesters, polyoxaesters, polyamidoesters, polyoxaesters containing amido groups, poly(anhydrides), polyphosphazenes, poly-α-hydroxy acids, trimethylene carbonate, poly-β-hydroxy acids, polyorganophosphazines, polyesteramides, polyethylene oxide, polyester-ethers, polyphosphoester, polyphosphoester urethane, cyanoacrylates, poly(trimethylene carbonate), poly(iminocarbonate), polyalkylene oxalates, polyvinylpyrolidone, polyvinyl alcohol, poly-N-(2-hydroxypropyl)-methacrylamide, polyglycols, aliphatic polyesters, poly(ester-amides), polyanhydrides, polysaccharides, proteins and mixtures thereof.

11. A medical device as in claim 8 further comprising a biodegradable plasticizer.

12. A medical device as in claim 11 wherein said plasticizer is selected from the group consisting of tributyl citrate, triethyl citrate, acetyltributyl citrate, and acetyltriethyl citrate; polyols, starches; vegetable oils; glucose or sucrose ethers and esters; polyethylene glycol ethers and esters; low toxicity phthalates; alkyl phosphate esters; dialkylether diesters; tricarboxylic esters; epoxidized oils; epoxidized esters; polyesters; polyglycol diesters; aliphatic diesters; alkylether monoesters; dicarboxylic esters; lecithin; and/or combinations thereof.

13. A medical device as in claim 1 wherein said balloon includes at least and inner balloon member and an outer balloon member.

14. A medical device as in claim 1 wherein said auxiliary functional structure comprises a cutting blade, force concentrator or a balloon stiffener mounted on the balloon.

15. A medical device as in claim 14 wherein said cutting blade, a force concentrator, or a balloon stiffener is formed of a polymer composition that is in a soft rubbery state at room temperature and that stiffens sufficiently to function effectively when cooled to said cryotreatment temperature.

16. A medical device as in claim 1 wherein the auxiliary functional structure is formed of a material composition that undergoes a glass transition between body temperature and said cryotreatment temperature.

17. A medical device as in claim 1 wherein the auxiliary functional structure is formed of a material composition that undergoes an increase in flexural modulus of about 15% or more between body temperature and the cryotreatment temperature.

18. A medical device comprising
a balloon and
a cryogen introduction system for introducing a cryogen into the balloon,
wherein the balloon comprises an auxiliary functional structure selected from the group consisting of a cutting blade, a force concentrator, or a stiffening member, said auxiliary functional structure being formed of a two-way shape memory material having a phase transition between two memory states that occurs at a temperature between body temperature and a cryotreatment temperature.

19. A medical device as in claim 18 wherein said two memory states comprises
a functional configuration when said balloon is at said cryotreatment temperature of said cutting blade, force concentrator, or stiffening member, respectively, for cutting a lesion, concentrating applied balloon force or stiffening the balloon, and
a body temperature configuration in which when the balloon is deflated said cutting blade, force concentrator, or stiffening member wraps around the deflated balloon.

20. A method of treating a subject body comprising
manipulating a device as in claim 1 to a treatment site within the subject body while the device is at body temperature,
cooling the device to a said cryotreatment temperature,
operating said auxiliary functional structure at the cryotreatment temperature,
returning the device to body temperature, and
removing the device from the subject body.
